(19) United States
(12) Patent Application Publication
(10) Pub. No.: US 2018/0156755 A1
(43) Pub. Date: Jun. 7, 2018

(54) APPARATUS FOR BIOMATERIAL AND METHOD OF DETECTING BIOMATERIAL

(71) Applicant: ELECTRONICS AND TELECOMMUNICATIONS RESEARCH INSTITUTE, Daejeon (KR)

(72) Inventors: Eun-Ju JEONG, Daejeon (KR); Bong Kyu KIM, Daejeon (KR); Kwang Hyo CHUNG, Daejeon (KR); Soo Jun PARK, Seoul (KR); Hyun Woo SONG, Daejeon (KR); Joo Yong SIM, Daejeon (KR); Chang-Geun AHN, Daejeon (KR); Chul HUH, Daejeon (KR)

(21) Appl. No.: 15/829,231

(22) Filed: Dec. 1, 2017

(30) Foreign Application Priority Data
Dec. 1, 2016 (KR) 10-2016-0163037
Nov. 14, 2017 (KR) 10-2017-0151763

(51) Int. Cl. G01N 29/036; G01N 33/543; C12Q 1/6809; C12Q 1/6806; G01N 27/02; C12Q 1/689; C12Q 1/686

(52) U.S. Cl. CPC G01N 29/036; G01N 33/54373; C12Q 1/6809; G01N 2291/0256; G01N 27/02; C12Q 1/689; C12Q 1/686; C12Q 1/6806

(57) ABSTRACT

Provided is an apparatus for detecting biomaterial and method of detecting biomaterial. The method include providing a sample including a fluorescent material and a biomaterial on one surface of an ultrasound receiving unit, and measuring an ultrasonic wave generated by the fluorescent material by emitting light to the sample.

### CROSS-REFERENCE TO RELATED APPLICATIONS

This U.S. non-provisional patent application claims priority under 35 U.S.C. § 119 of Korean Patent Application Nos. 10-2016-0163037 filed on Dec. 1, 2016, and 10-2017-0151763 filed on Nov. 14, 2017, the entire contents of which are hereby incorporated by reference.

### BACKGROUND

The present disclosure relates to biomaterial detection, and more particularly, to biomaterial detection using an acoustic signal.

Polymerase chain reaction (PCR) is a molecular biology technique that replicates and amplifies a desired portion of DNA. The PCR is highly complex and may selectively amplify only a specific fraction of DNA that a researcher desires in an extremely small amount of DNA solution. In addition, since the time required for amplification is as short as two hours and the experimental procedure is simple and may be amplified by a fully automatic machine, the PCR and various techniques derived therefrom treat DNA plays an extremely important role throughout the work such as molecular biology, medical treatment, criminal investigation, classification of organisms, and so on. According to the PCR, a primer corresponding to the first side of the target DNA portion in DNA used as a sample and a primer corresponding to the complementary chain at the end are chemically synthesized.

Fluorescent materials may be used in biomaterial detection. The wavelength of the light emitted from fluorescent materials may be detected by applying light to the fluorescent materials. However, the wavelength of the light (fluorescence) emitted from fluorescent materials overlaps with the wavelength of the excitation light, so that there is a limitation in detection accuracy. In addition, when a plurality of biomaterials are detected, interference between fluorescent materials is an issue.

### SUMMARY

The present disclosure provides a method of detecting biomaterial with improved detection efficiency and an apparatus used therefor.

An embodiment of the inventive concept provides a method of detecting biomaterial including: providing a sample including a fluorescent material and a biomaterial on one surface of an ultrasound receiving unit; and irradiating light onto the sample to measure an ultrasonic wave generated by the fluorescent material by emitting light to the sample.

In an embodiment, the method may further include performing a polymerase chain reaction of DNA, wherein measuring the biomaterial may be performed after performing the polymerase chain reaction, wherein the sample may includes the DNA.

In an embodiment, the fluorescent material may generate the ultrasonic wave by absorbing the light.

In an embodiment, the biomaterial may include a first biomaterial and a second biomaterial of different types, wherein the fluorescent material may include a first fluorescent material participating in a specific reaction of the first biomaterial and a second fluorescent material participating in a specific reaction of the second biomaterial.

In an embodiment, the fluorescent material may include: a first fluorescent material that absorbs light of a first wavelength and emits a first ultrasonic wave; and a second fluorescent material that absorbs light of a second wavelength and emits a second ultrasonic wave, wherein the second wavelength may be different from the first wavelength, wherein the second ultrasonic wave may have a different frequency from the first ultrasonic wave.

In an embodiment, the biomaterial may further include a third biomaterial different from the first and second biomaterials, wherein the fluorescent material may further include a third fluorescent material different from the first and second fluorescent materials and participating in a specific reaction of the third biomaterial, wherein the third fluorescent material may absorb light of a third wavelength and emit a third ultrasonic wave, wherein the third wavelength may be different from the first and second wavelengths, wherein the third ultrasonic wave may have a different frequency from the first and second ultrasonic waves

In an embodiment, the providing of the sample may include attaching the sample to the ultrasound receiving unit.

In an embodiment, the one side of the ultrasound receiving unit may include at least one of an upper surface and a side surface of the ultrasound receiving unit.

In an embodiment of the inventive concept, an apparatus for detecting biomaterial includes: a light source unit configured to emit light to a sample; and an ultrasound receiving unit disposed adjacent to the light source unit and configured to convert an ultrasonic wave emitted from the sample into an electrical signal.

In an embodiment, the ultrasonic wave may include a first ultrasonic wave and a second ultrasonic wave having different frequencies, wherein the ultrasound receiving unit may measure the first ultrasonic wave and the second ultrasonic wave simultaneously.

In an embodiment, the apparatus may further include a control unit configured to receive the electric signal from the ultrasound receiving unit.

### BRIEF DESCRIPTION OF THE FIGURES

The accompanying drawings are included to provide a further understanding of the inventive concept, and are incorporated in and constitute a part of this specification. The drawings illustrate exemplary embodiments of the inventive concept and, together with the description, serve to explain principles of the inventive concept. In the drawings:

FIG. 1 is a schematic diagram showing apparatus for detecting biomaterial according to embodiments of the inventive concept;

FIGS. 2A and 2B are diagrams for explaining a method of detecting biomaterial according to embodiments;

FIGS. 3A and 3B are schematic diagrams showing a polymerase chain reaction of a first biomaterial according to embodiments; and

FIGS. 4A and 4B are schematic diagrams showing apparatus for detecting biomaterial according to other embodiments.

### DETAILED DESCRIPTION

Hereinafter, preferred embodiments of the inventive concept will be described in detail with reference to the accompanying drawings. Advantages and features of the inventive concept, and implementation methods thereof will be clarified through following embodiments described with reference to the accompanying drawings. The inventive concept may, however, be embodied in different forms and should not be construed as limited to the embodiments set forth herein. Rather, these embodiments are provided so that this disclosure will be thorough and complete, and will fully convey the scope of the inventive concept to those skilled in the art and the inventive concept is only defined by the scope of the claims.

The terms used in this specification are used only for explaining specific embodiments while not limiting the inventive concept. The terms of a singular form may include plural forms unless referred to the contrary. The meaning of “include,” “comprise,” “including,” or “comprising,” specifies a property, a region, a fixed number, a step, a process, an element and/or a component but does not exclude other properties, regions, fixed numbers, steps, processes, elements and/or components. In addition, since they are in accordance with the preferred embodiment, the reference numerals shown in the order of description are not necessarily limited to the order. In addition, in this specification, when a film is referred to as being on another film or substrate, it may be directly formed on another film or substrate, or a third film may be interposed therebetween.

Although the terms first, second, third, etc. have been used in various embodiments herein to describe components (or structures) and the like, it should be understood that these regions and layers are not limited to the terms. These terms are merely used to distinguish predetermined components (or structures) from other components (or structures). Accordingly, the component referred to as a first structure in any one embodiment may be referred to as a second structure in other embodiments. Embodiments described herein include complementary embodiments thereof. Like reference numerals refer to like components throughout the specification.

An apparatus for detecting biomaterial (hereinafter referred to as a “biomaterial detection apparatus”) and a method of detecting biomaterial (hereinafter referred to as a “biomaterial detection method”) using the same according to the concept of the inventive concept will be described.

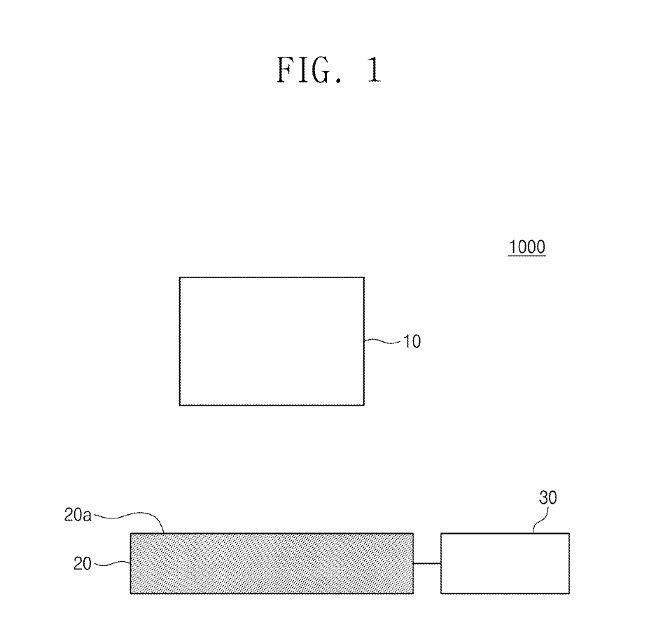

FIG. 1 is a schematic diagram illustrating a biomaterial detection apparatus according to embodiments of the inventive concept.

Referring to FIG. 1, a biomaterial detection apparatus 1000 may include a light source unit 10, an ultrasound receiving unit 20, and a control unit 30. A sample (not shown) containing biomaterials may be prepared. The sample may be provided on the ultrasound receiving unit 20. As an example, the ultrasound receiving unit 20 may serve as a receiver for receiving the photoacoustic signal generated from the biomaterial and as a support for the sample. As another example, a separate sample support (not shown) may be provided, and the ultrasound receiving unit 20 may be disposed on the sample support.

The light source unit 10 may be provided adjacent to the ultrasound receiving unit 20. For example, the light source unit 10 may be spaced from the upper surface 20a of the ultrasound receiving unit 20 on the upper surface 20a of the ultrasound receiving unit 20. The light source unit 10 may include a pulse laser. The light source unit 10 may irradiate light onto the sample. When the light is irradiated to the sample, the sample may emit an acoustic signal such as an ultrasonic wave. The ultrasound receiving unit 20 may function as an ultrasound transducer. The ultrasound receiving unit 20 may convert the ultrasonic wave emitted from the sample into an electrical signal. The ultrasound receiving unit 20 may further include a lock-in amplifier to amplify the received sound signal. As another example, the ultrasound receiving unit 20 may further perform the function of filtering the noise of the ultrasonic wave inputted from the sample. The control unit 30 may be provided adjacent to the ultrasound receiving unit 20. The control unit 30 may receive an electrical signal from the ultrasound receiving unit 20. The control unit 30 may perform a quantitative or qualitative analysis of the sample. Although not shown in the drawing, the biomaterial detection apparatus 1000 may further include a display unit. The display unit may display the analyzed result.

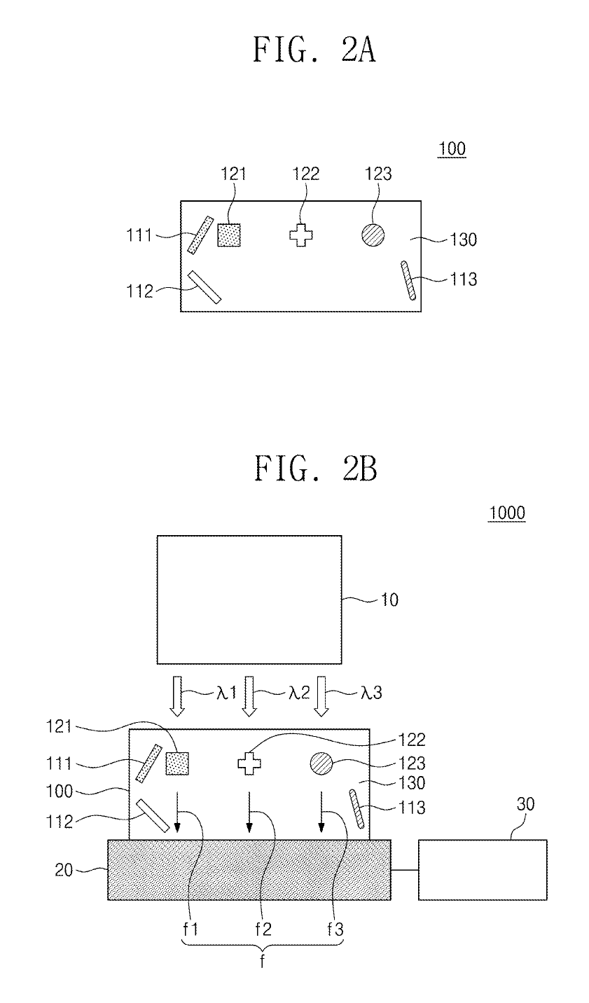

FIGS. 2A and 2B are views for explaining a biomaterial detection method according to the embodiments. Hereinafter, the contents overlapping with those described above will be omitted.

Referring to FIG. 2A, a sample 100 may be prepared. The sample 100 may include biomaterials 111, 112, and 113, fluorescent materials 121, 122, and 123, and a medium 130. The biomaterials 111, 112, and 113 may include a first biomaterial 111, a second biomaterial 112, and a third biomaterial 113. The biomaterials 111, 112, and 113 may be target materials to be analyzed. The biomaterials 111, 112, and 113 may be obtained from human or animal blood, urine, and saliva. The biomaterials 111, 112, and 113 may include nucleic acids, cells, viruses, proteins, and combinations thereof. When the biomaterials 111, 112 and 113 are proteins, the biomaterials 111, 112 and 113 may be one of an antigen, an antibody, a substrate protein, an enzyme, and a coenzyme. In addition, when the biomaterials 111, 112, and 113 are nucleic acids, they may include DNA, RNA, or hybrids thereof. The first to third biomaterials 111, 112, and 113 may be biomaterials for different diseases. In one example, the first biomaterial 111, the second biomaterial 112, and the third biomaterial 113 may be DNA for breast cancer, DNA for diabetes, and DNA for Alzheimer, respectively.

The fluorescent materials 121, 122, and 123 may include a first fluorescent material 121, a second fluorescent material 122, and a third fluorescent material 123, respectively. The first fluorescent material 121, the second fluorescent material 122, and the third fluorescent material 123 may be fluorescent materials of different kinds. The first fluorescent material 121, the second fluorescent material 122, and the third fluorescent material 123 may absorb light of a first wavelength region, light of a second wavelength region, and light of a third wavelength region, respectively. In this specification, light of a specific wavelength region may mean light of a pulse form having the specific wavelength as a center peak. For example, the light of the first wavelength region may be a pulse light having the first wavelength as a center peak, and may include light having a first wavelength and a wavelength adjacent to the first wavelength. The first wavelength, the second wavelength, and the third wavelength may be different from each other. The first fluorescent material 121 may be a fluorescent material for detecting the first biomaterial 111 and may be a fluorescent material participating in the specific reaction of the first biomaterial 111. The second fluorescent material 122 and the third fluorescent material 123 may be fluorescent materials for detecting the second biomaterial 112 and the third biomaterial 113, respectively. The second fluorescent material 122 and the third fluorescent material 123 may be fluorescent materials participating in the specific reaction of the second biomaterial 112 and the specific reaction of the third biomaterial 113, respectively. The specific reaction may include at least one of a polymerase chain reaction, an antigen-antibody reaction, an enzyme-linked immunospecific assay (ELISA) reaction, and an immune reaction.

The medium 130 may be in a liquid state. As an example, the medium 130 may be a solvent. The biomaterials 111, 112, and 113 and the fluorescent materials 121, 122 and 123 may be dispersed in the medium 130.

Unlike the illustrated example, the sample 100 may not include the third biomaterial 113 and the third fluorescent material 123. As another example, the sample 100 may not include the second biomaterial 112, the third biomaterial 113, the second fluorescent material 122, and the third fluorescent material 123. As another example, the sample 100 may further include a fourth biomaterial and a fourth fluorescent material (not shown).

The first to third biomaterials 111, 112, and 113 exist in a small amount in the living body, and thus, a small amount thereof may be obtained. In the analysis of the first to third biomaterials 111, 112, and 113, amplification of the first to third biomaterials 111, 112, and 113 may be required. For example, when the first to third biomaterials 111, 112, and 113 are DNA, a polymerase chain reaction of the first to third biomaterials 111, 112, and 113 may be performed. The case where the first to third biomaterials 111, 112, and 113 are DNA is exemplarily illustrated, but the biomaterials 111, 112, and 113 to be analyzed in the inventive concept are not limited to DNA. According to another embodiment, the first to third biomaterials 111, 112, and 113 may include enzymes, antigens or antibodies, and the detection method of the biomaterials 111, 112, and 113 may include performing the antigen-antibody reaction, enzyme-linked immunospecific assay (ELISA) reaction, or immune reaction of the biomaterials 111, 112, and 113 instead of polymerase chain reaction. Hereinafter, the polymerase chain reaction will be described.

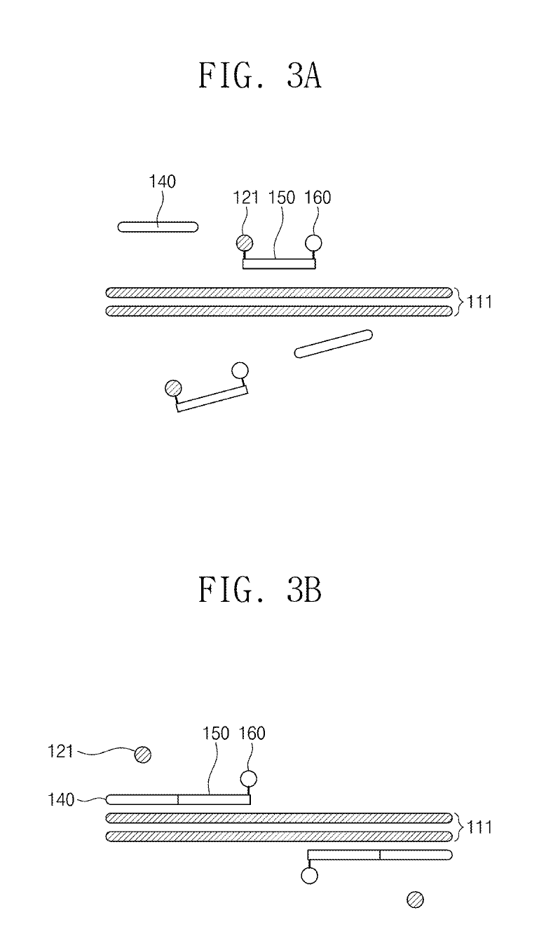

FIGS. 3A and 3B are diagrams schematically showing a polymerase chain reaction of a first biomaterial in a sample according to embodiments. Hereinafter, the contents overlapping with those described above will be omitted.

Referring to FIG. 3A, a primer 140, a DNA polymerase (not shown), a nucleotide 150, a first fluorescent material 121, and a quencher 160 are added to a first biomaterial 111 so that a sample 100 may be prepared. The first biomaterial 111 may include DNA. The nucleotide 150 may have a sequence complementary to the first biomaterial 111. The nucleotide 150 may serve as a dual labeled probe. If the first fluorescent material 121 is disposed with a less than predetermined interval from the quencher 160, the first fluorescent material 121 may be difficult to emit fluorescence by the quencher 160. According to embodiments, the first fluorescent material 121 may be coupled to one end of the nucleotide 150 and the quencher 160 may be coupled to the other end of the nucleotide 150. One end of the nucleotide 150 corresponds to the 5′ position, and the other end of the nucleotide 150 corresponds to the 3′ position. In this case, the first fluorescent material 121 coupled to the nucleotide 150 is disposed close to the quencher 160, so that the expression of fluorescence may be suppressed.

Referring to FIG. 3B, the polymerase chain reaction of the first biomaterial 111 may be performed to amplify the number of the first biomaterials 111. The polymerase chain reaction may be a real-time polymerase chain reaction. When the amplification of the first biomaterial 111 is in progress, the coupling between the nucleotide 150 and the first fluorescent material 121 may be broken by the DNA polymerase. The first fluorescent material 121 may be moved away from the quencher 160. Accordingly, the first fluorescent material 121 may emit fluorescence by light emission. The primer 140 and the nucleotide 150 may be used for amplification of the first biomaterial 111.

Although not shown in the drawing, the polymerase chain reactions of the second biomaterial 112 and the third biomaterial 113 may be performed in substantially the same manner as described in the example of the polymerase chain reaction of the first biomaterial 111. However, the polymerase chain reactions of the second and third biomaterials 112 and 113 may be performed specifically. For example, the primer, the DNA polymerase, the nucleotide, and the second fluorescent material 122 used in the polymerase chain reaction of the second biomaterial 112 may be different from the primer 140, the DNA polymerase, the nucleotide 150, and the first fluorescent material 121 used in the polymerase chain reaction of the first biomaterial 111. The primer, the DNA polymerase, the nucleotide 150, and the third fluorescent material 123 used in the polymerase chain reaction of the third biomaterial 113 may be different from those used in the polymerase chain reactions of the first biomaterial 111 and the second biomaterial 112. The polymerase chain reactions of the second biomaterial 112 and the third biomaterial 113 may be performed with the polymerase chain reaction of the first biomaterial 111 through a single process.

Referring to FIG. 2B, a sample 100 may be provided on the ultrasound receiving unit 20. The sample 100 may be disposed between the light source unit 10 and the ultrasound receiving unit 20. The sample 100 may contact the ultrasound receiving unit 20 directly or indirectly through another mediator. For example, the sample 100 may be provided in a beaker, and the beaker may be provided on the ultrasound receiving unit 20. As another example, the sample 100 may be prepared in the form of a gel or gel film, and the sample 100 may be attached on an ultrasound receiving unit 20. An adhesive film (not shown) may further be interposed between the sample 100 and the ultrasound receiving unit 20. The polymerase chain reaction described above in FIGS. 3A and 3B may be performed before or after the sample 100 is provided on the ultrasound receiving unit 20.

After the completion of the polymerase chain reactions of the first to third biomaterials 111, 112, and 113, the lights λ1, λ2, and λ3 may be irradiated onto the sample 100. The lights λ1, λ2, and λ3 may be lights by laser pulses. The lights λ1, λ2, and λ3 may include light λ1 of first wavelength region, light λ2 of second wavelength region, and light λ3 of third wavelength region. If the first to third biomaterials 111, 112 and 113 are present in the sample 100, after the polymerase chain reactions of the first to third biomaterials 111, 112, and 113 are performed, the first to third fluorescent materials 121, 122, and 123 may not be affected by the quencher (160 in FIG. 3B). The first to third fluorescent materials 121, 122 and 123 may absorb the lights λ1, λ2, and λ3, respectively and convert them into heats. The medium 130 in the sample 100 expands by heats, and an acoustic signal such as an ultrasonic wave may be generated. The first fluorescent material 121, the second fluorescent material 122, and the third fluorescent material 123 may generate a first ultrasonic wave f1, a second ultrasonic wave f2, and a third ultrasonic wave f3, respectively. In the specification, the generation of an ultrasonic wave by a fluorescent material involves the generation of an ultrasonic wave by the thermal expansion of the medium by the fluorescent material. The first ultrasonic wave f1, the second ultrasonic wave f2, and the third ultrasonic wave f3 may have a first frequency, a second frequency, and a third frequency, respectively. The first frequency, the second frequency, and the third frequency may be 50 Hz to 500 kHz. The first frequency, the second frequency, and the third frequency may be different from each other. The ultrasound receiving unit 20 may be in direct or indirect contact with the sample 100. Accordingly, the first ultrasonic wave f1, the second ultrasonic wave f2, and the third ultrasonic wave f3 may be measured by the ultrasound receiving unit 20. The measurement of the ultrasonic waves f1, f2, and f3 may be performed after the polymerase chain reaction. The ultrasound receiving unit 20 may output the first ultrasonic wave f1, the second ultrasonic wave f2, and the third ultrasonic wave f3 as electrical signals.

The electrical signals may be transmitted to the control unit 30. The control unit 30 may analyze the electrical signals to perform qualitative or quantitative analysis of the first to third biomaterials 111, 112, and 113.

The detection accuracy of the biomaterials 111, 112, and 113 may be restricted if the biomaterial detection method is performed by measuring the lights emitted from the first to third fluorescent materials 121, 122, and 123. For example, the first to third fluorescent materials 121, 122, and 123 may emit lights (not shown) in a fourth wavelength region, a fifth wavelength region, and a sixth wavelength region, respectively. Analysis of the first biomaterial 111 may be performed by sensing light of the fourth wavelength region. In this case, it is required that the fourth wavelength region is different from the first to third wavelength regions, the fifth wavelength region, and the sixth wavelength region. However, because the first wavelength region and the fourth wavelength region are partially overlapped, or the fourth wavelength region is completely separated from the first wavelength region, it is difficult to perform analysis. Accordingly, during the detection of the light of the fourth wavelength region, the light λ1 of the first wavelength region may act as noise. Similarly, during the analysis of the second biomaterial 112 and the third biomaterial 113, the light λ2 of the second wavelength region and the light λ3 of the third wavelength region may act as noise, respectively. In the process of detecting the light of the fourth wavelength region, at least one of the lights of the second wavelength region, the third wavelength region, the fifth wavelength region, and the sixth wavelength region may further serve as noise. In this case, it may be difficult to simultaneously analyze the plurality of biomaterials 111, 112, and 113.

According to the inventive concept, acoustic signals may have very different wavelengths and frequencies than optical signals. The first ultrasonic wave f1, the second ultrasonic wave f2, and the third ultrasonic wave f3 may have very different frequencies than the light λ1 of the first wavelength region, the light λ2 of the second wavelength region, and the light λ3 of the third wavelength region. Thus, in the process of analyzing the first to third biomaterials 111, 112, and 113, noise due to the light incident from the light source unit 10, for example, noise due to the lights λ1, λ2, and λ3 of the first wavelength region, the second wavelength region, and the third wavelength region, may be eliminated. Thus, the analytical accuracy of the biomaterials 111, 112, and 113 may be improved. In addition, the first ultrasonic wave f1, the second ultrasonic wave f2, and the third ultrasonic wave f3 may be easily separated from each other. According to embodiments, analysis of the various biomaterials 111, 112, and 113 may be performed simultaneously. The biomaterials 111, 112, and 113 may be analyzed quickly. In an embodiment, after the plurality of biomaterials 111, 112, 113 are obtained from blood, various diseases such as breast cancer, diabetes, and Alzheimer's may be analyzed simultaneously.

Since the analysis of the biomaterials 111, 112, and 113 is performed by receiving the ultrasonic waves f1, f2, and f3, the biomaterial detection apparatus 1000 may omit optical filters and a beam splitter (not shown) between the sample 100 and the control unit 30. At this time, the optical filters may mean filters that filter the lights λ1, λ2, and λ3 of the first to third wavelength regions. Accordingly, the biomaterial detection apparatus 1000 may be miniaturized.

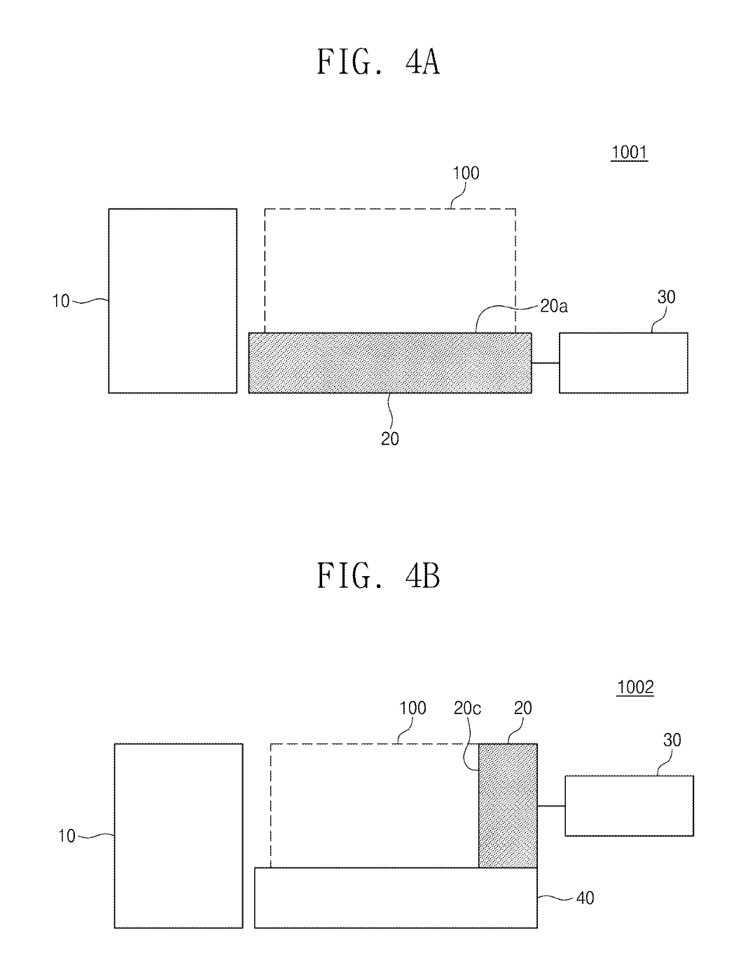

FIGS. 4A and 4B are schematic diagrams showing biomaterial detection apparatus according to other embodiments.

Referring to FIGS. 4A and 4B, a biomaterial detection apparatus 1001 or 1002 may include a light source unit 10, an ultrasound receiving unit 20, and a control unit 30. The light source unit 10, the ultrasound receiving unit 20, and the control unit 30 may perform substantially the same functions and roles as those described above with reference to FIG. 1. Through the biomaterial detection apparatus 1001 or 1002, a biomaterial detection method may be performed as described with reference to FIGS. 2A and 2B. However, the light source unit 10 and the ultrasound receiving unit 20 may be arranged in various ways.

Referring to FIG. 4A, the light source unit 10 may be disposed at one side of the ultrasound receiving unit 20. The sample 100 may be disposed on the upper surface 20a of the ultrasound receiving unit 20 as indicated by a dotted line.

Referring to FIG. 4B, the biomaterial detection apparatus 1002 may further include a sample support 40. The ultrasound receiving unit 20 may be disposed on the sample support 40. In this case, the sample 100 may be provided on the sample support 40 as indicated by a dotted line. The sample 100 may be placed on a side 20c of the ultrasound receiving unit 20.

The arrangement of the light source unit 10, the ultrasound receiving unit 20, and the control unit 30 may be variously modified without being limited to those shown in FIGS. 1, 4A, and 4B.

According to the inventive concept, biomaterials may be detected by measuring ultrasonic waves. The ultrasonic waves may be generated from fluorescent particles. As a result, biomaterials may be measured with high accuracy. Even though biomaterials are different types of biomaterials, they may be easily measured at the same time.

The biomaterial detection apparatus according to embodiments does not include optical filters and beam splitter, and thus may be miniaturized.

Although the exemplary embodiments of the inventive concept have been described, it is understood that the inventive concept should not be limited to these exemplary embodiments but various changes and modifications can be made by one ordinary skilled in the art within the spirit and scope of the inventive concept as hereinafter claimed.

What is claimed is:

1. A method of detecting biomaterial comprising:
providing a sample including a fluorescent material and a biomaterial on one surface of an ultrasound receiving unit; and
irradiating light onto the sample to measuring an ultrasonic wave generated by the fluorescent material.

2. The method of claim 1, further comprising performing a polymerase chain reaction of DNA,
wherein measuring the biomaterial is performed after performing the polymerase chain reaction,
wherein the sample comprises the DNA.

3. The method of claim 1, wherein the fluorescent material generates the ultrasonic wave by absorbing the light.

4. The method of claim 1, wherein the biomaterial comprises a first biomaterial and a second biomaterial of different types,
wherein the fluorescent material comprises a first fluorescent material participating in a specific reaction of the first biomaterial and a second fluorescent material participating in a specific reaction of the second biomaterial.

5. The method of claim 4, wherein the fluorescent material comprises:
a first fluorescent material that absorbs light of a first wavelength and emits a first ultrasonic wave; and
a second fluorescent material that absorbs light of a second wavelength and emits a second ultrasonic wave,
wherein the second wavelength is different from the first wavelength,
wherein the second ultrasonic wave has a different frequency from the first ultrasonic wave.

6. The method of claim 5, wherein the biomaterial further comprises a third biomaterial different from the first and second biomaterials,
wherein the fluorescent material further comprises a third fluorescent material different from the first and second fluorescent materials and participating in a specific reaction of the third biomaterial,
wherein the third fluorescent material absorbs light of a third wavelength and emits a third ultrasonic wave,
wherein the third wavelength is different from the first and second wavelengths,
wherein the third ultrasonic wave has a different frequency from the first and second ultrasonic waves

7. The method of claim 1, wherein the providing of the sample comprises attaching the sample to the ultrasound receiving unit.

8. The method of claim 1, wherein the one side of the ultrasound receiving unit comprises at least one of an upper surface and a side surface of the ultrasound receiving unit.

9. An Apparatus for detecting biomaterial comprising:
a light source unit configured to irradiate light to a sample; and
an ultrasound receiving unit disposed adjacent to the light source unit and configured to convert an ultrasonic wave emitted from the sample into an electrical signal.

10. The apparatus of claim 9, wherein the ultrasonic wave comprises a first ultrasonic wave and a second ultrasonic wave having different frequencies,
wherein the ultrasound receiving unit measures the first ultrasonic wave and the second ultrasonic wave simultaneously.

11. The apparatus of claim 9, further comprising a control unit configured to receive the electric signal from the ultrasound receiving unit.
